(19) United States
(12) Patent Application Publication
(10) Pub. No.: US 2024/0180664 A1
(43) Pub. Date: Jun. 6, 2024

(54) INSTRUMENT KIT TRACKING SYSTEM WITH PARENT-CHILD FEATURES

(71) Applicant: Q Med Innovations, Inc., Middletown, RI (US)

(72) Inventors: Shelby Kornbluth, Foxboro, MA (US); Victor Nunes, Middletown, RI (US); James Calvin, Attleboro, MA (US); David Tyree, Warwick, RI (US); Ken Doucette, Norton, MA (US)

(73) Assignee: Q Med Innovations, Inc., Middletown, RI (US)

(21) Appl. No.: 18/526,164

(22) Filed: Dec. 1, 2023

Related U.S. Application Data
(60) Provisional application No. 63/385,784, filed on Dec. 2, 2022.

(51) Int. Cl. A61B 90/90; G16H 40/40

(52) U.S. Cl. CPC A61B 90/90; G16H 40/40; A61B 2090/0805

(57) ABSTRACT

The present disclosure is directed to an instrument kit tracking system that includes a housing that has a parent tracker affixed thereto as well as at least one medical instrument tray residing in the instrument kit housing that receives at least one medical instrument. A child tracker affixed to each of the at least one medical instrument tray where the child tracker communicates data to the parent tracker to obviate the need for full communications electronics, such as including GPS, within the child tracker to thereby save costs while sacrificing minimal loss of accuracy. The temperature sensor in the parent tracker records a temperature reading and calculate a usage of the at least one medical instrument if the temperature reading is above a predetermined value.

### CROSS REFERENCE TO RELATED APPLICATIONS

This application claims benefit of U.S. Provisional Patent Application No. 63/385,784, filed Dec. 2, 2022, the entire contents thereof are incorporated by reference.

### BACKGROUND OF THE INVENTION

The present disclosure generally relates to the medical industry, specifically to medical instrument kits as well as the object tracking industry, including tracking systems for medical instrument kits.

In the medical industry, current instrument kits suffer from the inability to properly track such instrument kits once they are deployed to the sales force network or hospitals. These instrument kits are often assets of the original equipment manufacturers (OEM) and often placed on loan to a hospital to support a surgical procedure. One issue with such kits is, once they are out of the OEM's control, the sets are often misplaced, lost, or simply contained within the sales network without further visibility by the OEM. These lost, misplaced, or otherwise disposed kits come at an extreme cost to the OEM's each year.

Additionally, there is a concern in the industry and by the FDA as to how instruments are being managed. Such management can include the calibration, repair, or ultimately replacement of the instruments once they are deployed to support surgery. Current instrument kits fail to provide a way of tracking whether a kit has been in one or a hundred surgeries. As such, the prior art systems fail to provide the OEM, hospitals, or other users the current lifecycle of the kits or the instruments contained therein.

Solutions to address known problems in the industry have been primarily software solution, RFID which required human intervention or Bluetooth which requires infrastructure. For example, there have been attempts to track surgical assets with software applications and phone apps to facilitate the management of the location of these sets and replenishment of implant or instrument inventory. These efforts are inadequate and suffer from inaccuracy and lack of integrity because these tools require manual input and many times data is corrupted due to the lack of input given to the systems on a timely basis.

For example, there is a particular need in the industry for the ability to not only track an entire instrument kit but to also separately track and monitor any tray within that instrument kit.

### SUMMARY OF THE INVENTION

Thus, the instrument kit tracking system and methods of the present invention can provide the ability to monitor and report on a daily basis the exact location of each kit without human interaction. Moreover, the instrument kit tracking system and method of the present invention can record and transmit, within the same daily schedule, all date stamps, locations, and number of surgeries the kit has been involved in. A cloud-based system can be included to capture data on a daily basis and report out to the user or manager the particular status of each deployed kit. The cloud-based system can additionally alert end users of any kit that may have reached a predefined limitation on the number of surgeries performed so that the kit can be returned and instruments replaced or calibrated before going back out for deployment. The instrument kit tracking system and method of the present invention offers greater efficiency in inventory management, deployment, accountability of the assets, automatic annual inventory auditing, complete compliance to instrument usage and documentation, and ease of inventory recovery during product recalls.

There have been attempts in the prior art to address the foregoing known problems in the industry. For example, there have been attempts to track surgical assets with software applications and phone apps to facilitate the management of the location of these sets and replenishment of implant or instrument inventory. These efforts are inadequate and suffer from inaccuracy and lack of integrity because these tools require manual input and many times data is corrupted due to the lack of input given to the systems on a timely basis.

For the foregoing reasons, an object of the present invention is to provide a system and method for continuous tracking of surgical, or medical kits, that can continuously track even during multiple high temperature sterilization processes.

A further object of the present invention is to provide the ability to not only track an entire instrument kit but to also separately track and monitor any tray within that instrument kit.

### BRIEF DESCRIPTION OF THE DRAWING FIGURES

The novel features that are characteristic of the present disclosure are set forth in the appended claims. However, the disclosure's preferred embodiments, together with further objects and attendant advantages, will be best understood by reference to the following detailed description taken in connection with the accompanying drawings in which:

FIG. 1 is perspective view of a medical kit according to a first embodiment;

FIG. 2 is a sectional elevational view through the line 2-2 of the instrument kit tracking system of FIG. 1;

FIG. 3 is a close-up sectional view of the embodiment of the present invention that employs a thin layer of material adhered directly to the inner surface of the case housing that is embedded in the instrument kit;

FIG. 4 is a close-up sectional view of the embodiment of the present invention employing a sock-like inner enclosure to house the circuit board electronics in the case housing;

FIG. 5 is a second embodiment of the present invention where the location tracking electronics is contained within a separate electronics module that is secured to an instrument kit, such as by rivets, or the like;

FIG. 6 is a close-up cross-sectional view through line 6-6 of the electronics module of FIG. 5 where the case housing includes the thin layer adhered to the inner surface of the case housing;

FIG. 7 is a close-up cross-sectional view showing another version of the standalone electronics case housing to include sock-like inner enclosure to house the circuit board electronics in the case housing;

FIGS. 8A and 8B show additional embodiments of the standalone electronics module where the circuit board is vacuum sealed;

FIG. 9 is showing yet another embodiment of an electronics module that includes a layer of insulation;

FIG. 10 is a side view of the standalone module of FIG. 5, containing the tracking electronics therein, affixed to the side of an instrument kit, such as by rivets;

FIG. 11 an exploded perspective view of yet another embodiment of the instrument kit tracking system of the present invention that includes parent and child components that cooperate together to enable separate tracking of any given tray within the instrument kit as well as the instrument kit, as a whole; and

FIG. 12 is an exploded side elevational view of embodiment of the instrument kit tracking system of the present invention shown in FIG. 11.

### DESCRIPTION OF THE INVENTION

Certain exemplary embodiments will now be described to provide an overall understanding of the principles of the structure, function, manufacture, and use of the device and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present disclosure. Further, in the present disclosure, like-numbered components of the embodiments generally have similar features, and thus within a particular embodiment each feature of each like-numbered component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. Further, to the extent that directional terms like proximal, distal, top, bottom, up, or down are used, they are not intended to limit the systems, devices, and methods disclosed herein. A person skilled in the art will recognize that these terms are merely relative to the system and device being discussed and are not universal.

In the prior art, it is well known to bundle common surgical instruments which are required for a particular type of surgical procedure together in specialized kits. There are numerous health and safety requirements for these kits including tight inventorying and sterilization. Current systems rely on manual recording of the particular kits by serial number, the number of uses of each kit, and the locations of such uses. However, current systems lack the ability to automatically track the location and uses of each kit, notify end users of that data, and continue to provide that data even during high temperature sterilization processes.

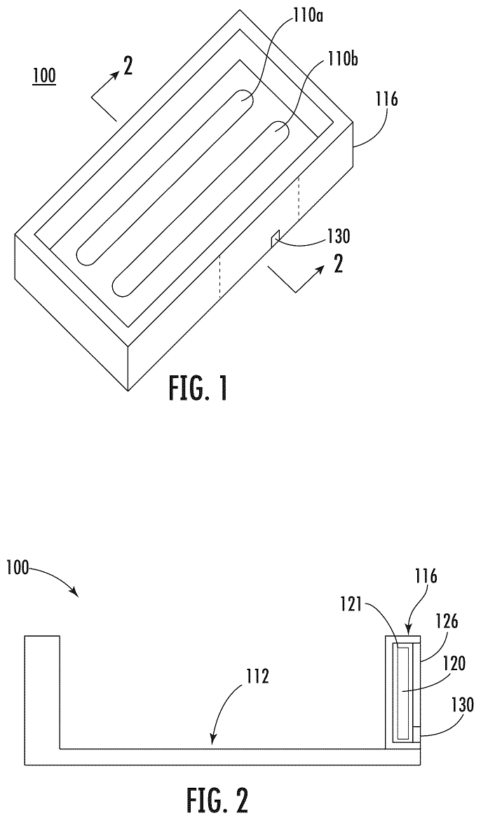

As shown in FIGS. 1 and 2, a first embodiment of a surgical kit 100 can include one or a plurality of medical instruments, or tools 110a, 110b. An example of such an instrument kit is 100 shown in FIG. 10. The kit 100 can be in the form of a tray 112, as representationally shown, or can be in the form of a bag or other types of containers. The tray 112 can be made of any material that is capable of being sterilized, including but not limited to stainless steel and other metals. The tray 112 can include tool supports or cavities to retain the tools 110a, 110b relative to one another to avoid any damage thereto. Moreover, as shown in FIG. 10, a tray 212 can include a cover 214 of any type to protect the tools 110a, 110b from the outside environment after sterilization in an autoclave, not shown.

An electronic tracking device 120 can be included on the kit 100, disposed in a side wall 116 of the tray, as shown in the embodiments of FIGS. 1-4, or on a side wall 216 of the tray 200 as shown in FIGS. 5 and 10. The tracking device 120 can be any number of tracking devices. For example, the tracking device 120 can include a power source 122 and a temperature sensor 124. The temperature sensor 124 can record temperature changes that are likely to occur in an autoclave during sterilization, as discussed below. The temperature sensor 124 is preferably solid state but can be any type of temperature sensor and one that can sense temperatures up to and beyond temperatures used for sterilization by known sterilization equipment.

While not shown, the tracking device can alternatively or additionally, include a processor; a memory: a one-way or two-way communications module, e.g. BLUETOOTH, cellular connections; and/or at least one type of location sensor including GPS, BLUETOOTH, WI-FI, Cellular Triangulation or other types of location sensors. Such communications components are so well known that they do not need to be shown or discussed further herein. Advantageously, such communication modules

In some embodiments, the power source 122 can be sized to maintain functionality for five or more years and for more than 500 sterilization cycles. For example, the power source 122 can be any kind of battery, rechargeable or not. Advantageously, the electronic tracking device 120 can be permanently attached to either the inside or the outside of the tray (such as by rivets 229, adhesive, brackets or other fasteners as in FIG. 10) such that it cannot be removed intentionally or accidentally. In the illustrated embodiment, the electronic tracking device 120 can be disposed within a locked compartment 121 having a door 126 with a lock 130, such as seen in FIGS. 1-4. In an alternative embodiment, the compartment 121 can be similar to a drawer which can be withdrawn as a unit from the tray. The compartment can be locked by means of a mechanical lock 130 and key, an electronic locking mechanism, or a magnetic locking system, disposed on the door.

As noted above, the trays 112 and instruments 110a, 110b all need to be sterilized before and after use in a medical procedure. One such sterilization process, such as one that is carried out using an autoclave, subjects the tray 112, and thus the electronic tracking device 120, to being washed in an ultrasonic water bath solution and then steam sterilized at high temperatures around 120° C. to 135° C. in an autoclave. In some examples, the high temperatures can exceed 135° C.

Since the aforementioned electronics 126 typically operation in the range of 85° C., the present invention provides the necessary housing thereabout to protect the electronic during sterilization that far exceeds the operating temperature of the electronics. Moreover, the tray 112 of the instrument kit tracking system and method of the present invention can be designed to survive over 500 cycles of steam sterilization without destroying the electronic tracking device disposed therein. For example, the locked compartment 121 containing the electronic tracking device 120 can be designed to protect the tracking device from the aforementioned steam sterilization temperatures. As noted above, the tray/housing 112 can include a temperature sensor 124 can record such temperature changes and indicate a usage after the sensor has recorded a predefined high temperature setting that is associated with a sterilization through an autoclave. In some embodiments, the electronics 123, including the temperature sensor 125 and the power source 122 can be designed to withstand autoclave temperatures, discussed above, without any insulation such that they do not fail. The electronics 123 can include a memory to record these temperature changes as a usage and a processor to calculate the number of uses. Additionally, or alternatively, the electronics 123 can include a communications module to wirelessly communicate the temperature changes to an external CPU module, e.g. a smart phone, tablet, or a computer. In some embodiments, the communications modules can be programed such that they can automatically, without additional instructions, communicate the recorded temperature change, as a use, to as third-party device. In some embodiments, the recorded temperature change can additionally include location data which can be provided by the electronic 123, including GPS, BLUETOOTH, WI-FI, Cellular Triangulation or other types of location sensors. Advantageously, the instant tracking device 120 can provide real, or near real, time use and location data for the instrument tray without any additional interaction by users.

The electronic tracking device 120 can be defined by a compartment housing 140 that itself is preferably made of a polymer material but can be any suitable material. The housing 140 can be constructed to include a thin layer of material 142 that can be both hydrophobic and breathable in nature. For example, the hydrophobic layer may be epoxy, silicone, polyimide or silica aerogel. In some embodiments, the thin layer of material 142 can have a thickness of 6-10 mm but could be any thickness, such a thickness less than 6 mm, such as 1 mm, or greater than 10 mm. Moreover, the materials can be chosen to shield the electronics from the above noted autoclave temperatures. This layer of material 142 can work in conjunction with the actual polymer case 140 material type and can be designed to provide an air chamber around the electronics to provide an additional thermal barrier. This is one example of such a compartment, any compartment construction capable of permitting electronic signals to pass therethrough while protecting the internal electronics from autoclave temperatures is deemed to be within the scope of this disclosure.

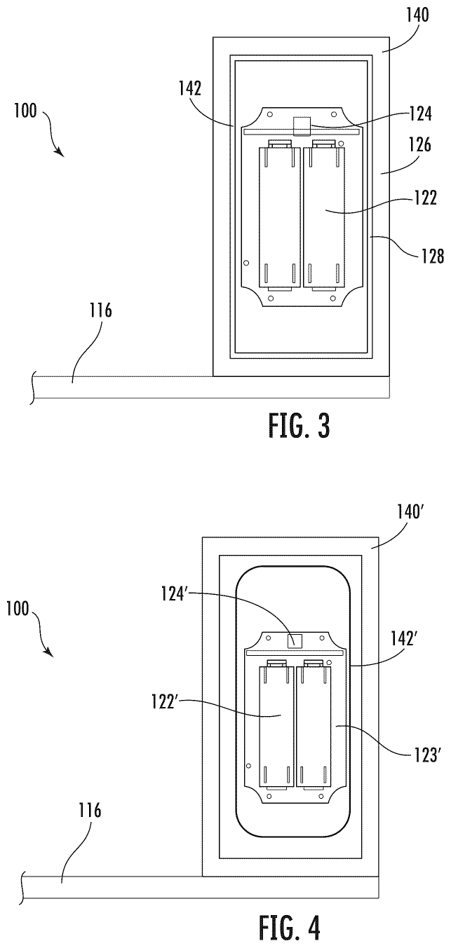

Further, it is envisioned that the thin layer material 142 can be located on the inside of the polymer housing 140 and affixed thereto, such as by glue or adhesive to the polymer case surrounding the electronics, as in FIG. 3. Alternatively, as in FIG. 4, the thin layer 142′ can be constructed as a sock or insert where the circuit board assembly 123′ (including the power source 122′ and at least a temperature sensor 124′) is slipped into the sock then placed inside the polymer case housing 140′.

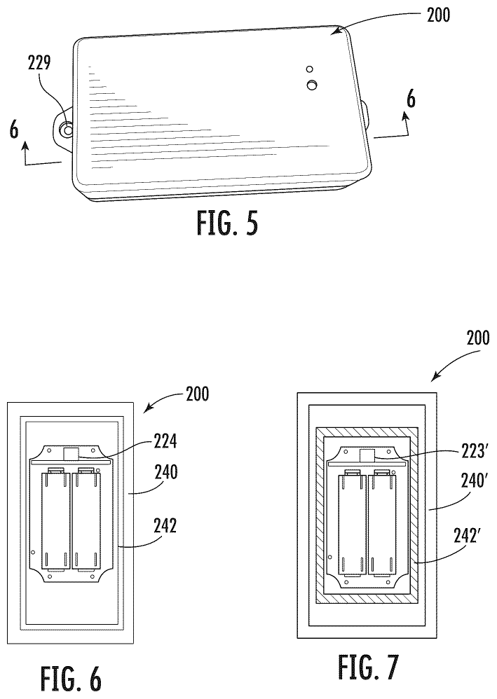

A standalone electronics module 200, that is affixed by rivets and the like, is shown in a second embodiment of FIGS. 5-7 and 10. In this second embodiment of the present invention, the location tracking electronics 223 are contained within a separate standalone electronics module 200 that is secured to an instrument kit 212/214, such as by rivets 229, or the like via mounting tabs on the case housing 240 of the module 200. Similar to the first embodiment, the standalone case housing 240 preferably includes an additional thin layer of material 242, which is preferably hydrophobic and also breathable. FIG. 6 shows a cross-sectional view through a version of the electronics module 200 of FIG. 5 where the case housing 240 inside includes the thin layer of material 242 adhered to the inner surface of the case housing 240. Alternatively, the thin layer 242 can be constructed as a sock or insert where the circuit board assembly 223′ is slipped into the sock then placed inside the polymer case housing 240. Of note, the housing 240 and the thin layer of material 242 can be substantially similar to the materials described above with respect to the housing 140 and thin layer of material 142. FIG. 7, shows an alternative version of the standalone electronics case 200′ housing that includes a sock-like inner enclosure 242′ to surround the circuit board electronics 223′ within the case housing 240′. The sock-like inner enclosure is preferably silica aerogel, glass fiber, or silicone fiber, but may be any other suitable material.

FIG. 10 shows a side view of the standalone case housing 200, containing the tracking electronics therein, affixed to the side of an instrument kit 214, preferably by the rivets 229. The housing 200 is preferably plastic or any other material, such as Polypropylene or polyphenylsulfone, that is suitable for withstanding high heat during an instrument sterilization process. Any other attachment fasteners can be employed and still be within the scope of the present invention. Adhesives and other fasteners may also be used to secure the electronics module 200 to the side 212 of the instrument kit 214. It is also preferred that the tracking device 200, including its electronics 223′, be controllably affixed to the instrument kit 214 for tamper proof and security purposes. For example, a special latching mechanism, or lock, 230 may be employed for this purpose to attach and detach the tracking device from the instrument kit with a special key. For example, the electronics case module 200 can be attached by rivets or other mechanism such adhesive or other types of fasteners. The electronics module 200 can be attached using a releasable security lock 230 where the electronics module can be attached and detached from the instrument kit 214 but only by use of the special key. In the embodiment of FIGS. 1-4, the access door 126 could be secured by a lock 130 with a special key.

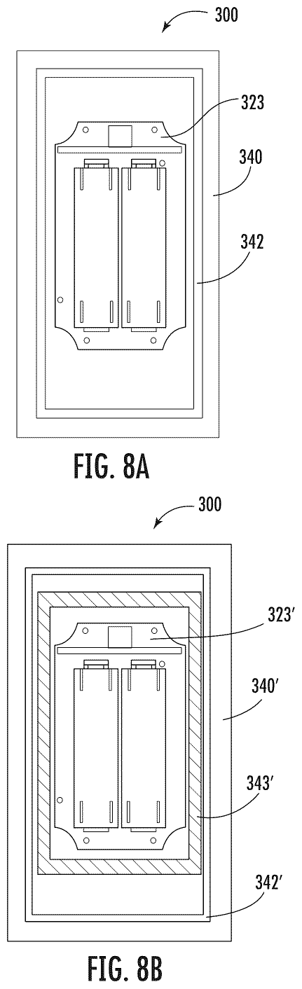

In a third alternative embodiment of the present invention of FIGS. 8A and 8B, in place of the thin layer of material 142, 242 affixed to an inner surface of the respective housings, or the sock like thin layer of material 142′, 242′ disposed around the respective electronics, a layer of material 342 is vacuum sealed around the electronics 323, as shown in FIG. 8A. FIG. 8B, shows a cross-sectional view showing another version of the standalone electronics case housing 340′ that includes an inner liner 343′ to surround the circuit board electronics 323′ within the vacuum sealed material 342′ in the case housing. Any combination of the foregoing layers and liners may be used and still be within the scope of the present invention.

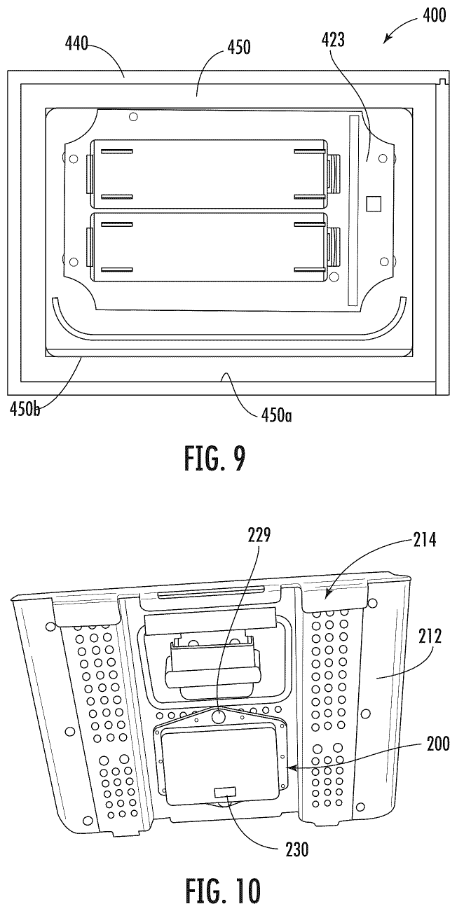

FIG. 9 illustrates a further alternative embodiment 400 of the present invention that includes an additional feature that provides a layer of insulation 450 between the housing 440 and the circuit board electronics 423. The insulation 450 is preferably silica aerogel but may be any suitable material depending on the application at hand. It is understood that the insulation 450 of FIG. 9 can be used in any of the above configurations without departing from the scope of the embodiments, including an inner layer of the thin material 142 in FIG. 3, the sock like embodiment 142′ of FIG. 4, or the vacuum sealed version 342 of FIG. 8A. In the case where the circuit board electronics 423 includes a temperature sensor 224 as seen in FIG. 6, for example, the high temperatures recorded during an autoclave disinfecting wash may be lower than those recorded in a sensor 224 without the insulation 450. A thin layer of the same material as the thin layer 142 can be use on an outer 450a or inner 450b surface of the insulation, or both. The inner layer 142 may or may not be used depending on the application at hand.

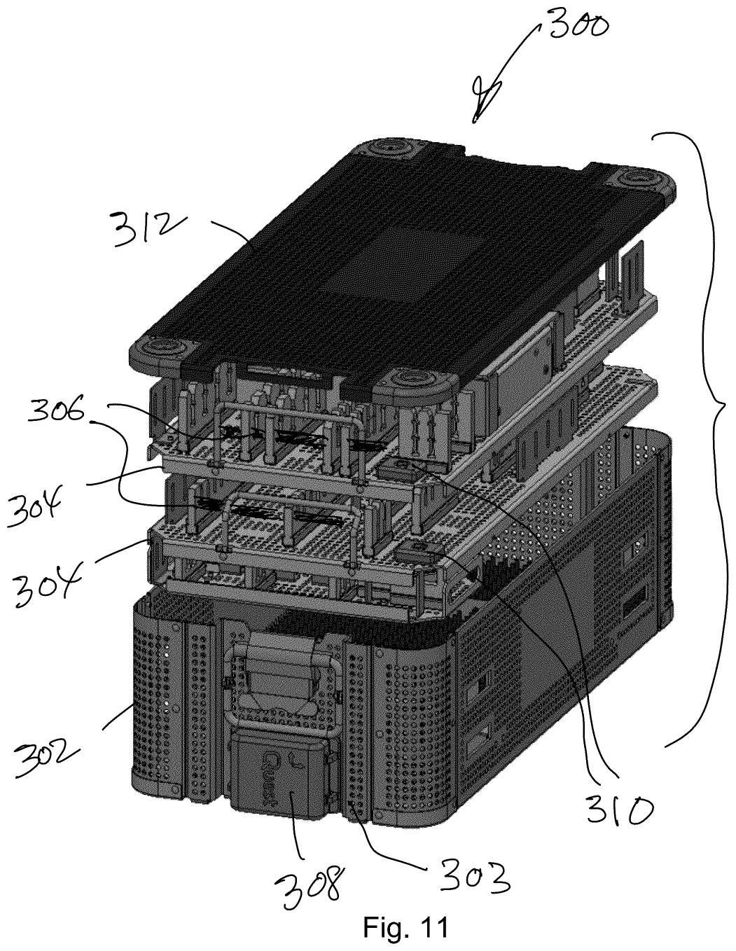

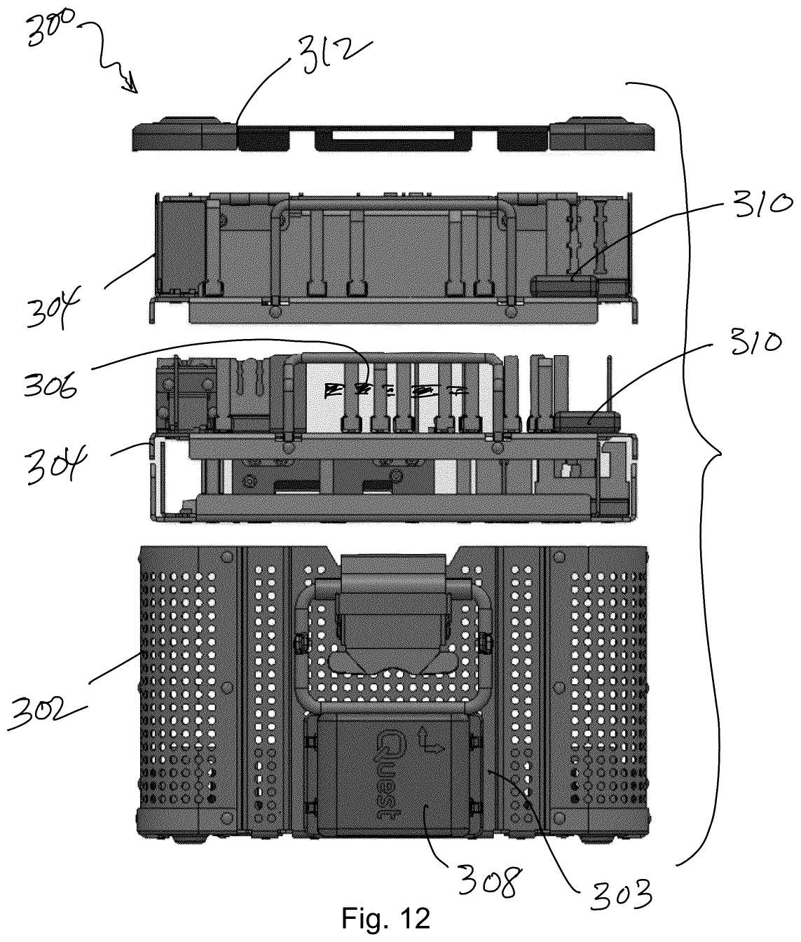

Referring now to FIGS. 11 and 12, yet another embodiment 300 of the present invention is shown in detail. This further embodiment provides an additional level of tracking and monitoring compared to the embodiments of FIGS. 1-10 which provide tracking and monitoring of the entire instrument kit, as a whole. In contrast, the embodiment of FIGS. 11 and 12 show an instrument kit tracking system 300 that not only tracks the entire instrument kit 300, as a whole, but also the individual trays 304 therein that are carrying medical instruments 306 therein.

The embodiment of FIGS. 11 and 12 carry out this further functionality of whole kit 300 and individual tray tracking and monitoring by providing a parent/child topology for the tracking units. For example, an instrument kit tracker, or “parent tracker” 308, is placed on the inside or the outside of an instrument kit housing 302. As seen in FIG. 11, the parent tracker 308 is affixed to a side panel 303 of the instrument kit 302, in similar fashion to the way the electronics module/tracker 200 is affixed to the side of the instrument kit housing, as seen in FIG. 10, using a security lock, adhesive, fasteners, and the like. Any type of fastener or method of affixation is envisioned in accordance with the present invention. Thus, the parent tracker 308 may be used to track and monitor the location of the instrument kit 300, as a whole.

“Child trackers” 310 are similarly placed on each tray 304 that is inside the instrument kit housing 302. In the example of FIGS. 11 and 12, two separate and discrete trays 304 are stackable on each other so they can reside inside the housing 302 of the instrument kit 300. A top cover 312 contains the stacked trays 304 within the instrument kit 300. Thus, each of the two trays 304, in this example, can be tracked and monitored separate and apart from each other as well as separate and apart from the overall instrument kit itself, namely, the outer housing 302. The tracking and monitoring of each of the trays 304 as well as the overall instrument kit 300 provides discrete information as to each of the trays 304 and the overall instrument kit 300. This is particularly helpful when a given tray 304 becomes separated from the other trays 304 that are being carried in the same instrument kit housing 302. This is common during surgeries where the instrument kit 300 is opened quickly for urgent use of a given instrument therein. Furthermore, after a given surgery, it is possible that the trays 304 are not reinstalled into the instrument kit housing 302 that they originally traveled in. This become even more problematic if more than one instrument kit 300, with multiple trays 304 therein, is used during a given surgery event.

The child trackers 310 collects surgical event data and location and transmits the data to the parent tracker 308. The parent tracker 308 receives the data from the child trackers 310 and transmits the data to the cloud. The child trackers 310 may be placed inside a solid wall sterilization container, such as an Asculap container, and transmit to the parent tracker 308. As shown in FIGS. 11 and 12, the child trackers 310 are respectively mounted to a surface of the trays 304, such as the floor thereof, as in the example shown. However, the child trackers 310 may be mounted to any surface of their respective tray 304. It is preferred that each tray 304 residing within the instrument kit 300 be equipped with a child tracker 310. The child trackers 310 can communicate with its parent tracker 308 or any other parent tracker 308, such as a “step parent.”

The child trackers 310 (which can be called “QuestTags”) on individual trays transmit data to its parent tracker 308 (which can be called a “Quest”) on the instrument kit 300 into which the individual trays 304 are install. Or, the child trackers 310 can transmit to the closest parent tracker 308 in its vicinity (not shown). The parent tracker 308 then transmits the data to a portal, in similar fashion to the way the tracker of FIGS. 1-10 transmits data back to a portal or cloud.

It is possible that multiple parent trackers 308 may transmit data from each child tracker 310. Once the data is transmitted to a portal, the location of each child tracker 310 is determined based on the location from the closest parent tracker 308. The location of each child tracker 310, and thus the location of the tray 304 associated with that child tracker 310, is displayed in the portal. In instances where a child tracker 310 is not closest to the parent tracker 308, and the closest parent tracker 308 is part of a defined set of “step parents” (such as the parent trackers assigned to a specific company), the portal will display the parent tracker 308 where the child tracker 310 is located and identify that it is with a “step parent”. Nevertheless, the location of the child tracker 310 and the associated tray 304 can be tracked.

In instances where a child tracker 310 is not closest to the parent tracker 308 and the closest parent tracker 308 is not part of a defined set of parent units (such as the parents are assigned to a different company), the portal will display the location and will identify in the system that the child tracker 310 is not with its preset associated parent tracker 308.

The topology of the parent tracker 308 and child trackers 310 obviates the need to provide full expensive GPS and other communication electronics in each and every child tracker 310 in order to track and monitor the location of individual trays 304 within an instrument kit 300. Thus, the child trackers 310 can be configured with electronics that do not have the range or capability of the parent trackers 308 to reduce the cost of the child trackers 310. The electronics in both the parent tracker 308 and child trackers 310 include a power supply (such as batteries, rechargeable batteries), a microprocessor with an operating system thereon as well a communications electronics, such a Wi-Fi, Bluetooth, and the like. However, the child trackers 310 would not need as large of a power supply as the parent tracker 308 because it does not include power demanding GPS.

Therefore, the ability of the child trackers 310 to communicate locally, in very close proximity to a parent tracker 308, is often sufficient to track and monitor the location of an individual trays 304 where there the location of the parent tracker 308 is used to estimate the location of child trackers 310 affixed to individual trays 304. In other words, precise location of the individual trays 304 is not critical where its approximate location can be determined when a parent tracker 308 reports that a given child tracker 310 is close by. This tradeoff of losing pinpoint location accuracy to greatly reduce cost is typically acceptable where approximate but very close location of the individual trays 304 and the child trackers 310 thereon is still enables recovery of any of the individual trays 304 after a surgery is complete.

The sterilization using an autoclave and use of an instrument kit tracking system 300 that includes the parent and child tracking features is generally similar to the tracking system of FIGS. 1-10 but with some additional accommodations due to the use of two different types of trackers 308, 310 that work together.

For example, once a parent tracker 308 or a child tracker 310 detects an elevated temperature, such as 60° C., it starts recording the temperature at specific intervals, such as 3 minutes or 10 minutes, until it reaches a high temperature, such as 105° C. When it reached 105° C. as in an autoclave cycle, temperature recording is stopped. Once the temperature returns to a temperature lower than 105° C., temperature recording resumes until the temperature reaches 40° ° C. Once the temperature reaches 40° C., the parent tracker 308 transmits location and temperature data to the cloud.

Also, if a parent tracker 308 or child tracker 308 detects the elevated temperature, such as 60° C., but never reaches the high temperature of 105° C., as in a wash or decontamination cycle from an autoclave or the like, the temperature continues to record at the predefined interval until the low temperature of 40° C. is reached and the location and temperature data is transmitted to the cloud.

Once the temperature data is received in the portal, and algorithm is run against the temperature data to determine if a wash or an autoclave cycle has occurred, which is then displayed in the portal. A second algorithm is run to determine if a surgical event has occurred. A surgical event is determined by using an autoclave, which is required prior to surgery, followed by a wash or decontamination cycle, which is required post-surgery, in the same physical location, such as a hospital. As a result, surgical event information can be used to determine usage of instrument kits 300 and in instances medical instrument items 306 in the instrument kit 300 require calibration after a specific number of surgeries. The instrument kit tracking system 300 of the present invention provides individual tracking of the trays 304 within the instrument kit 300 without incurring the high cost of providing standalone communications electronics in each child tracker 310. This also saves on maintenance of the child trackers 310. Since they do not have their own full standalone communications electronics, the amount of power needed to operate the child trackers 310 is much less than the parent tracker 308 saving further cost and maintenance in connection with power supplies, namely, batteries therein.

The instrument kit tracking system 300 and methods of the present invention provide the ability to periodically monitor and report, such as on a daily basis, but could be any pre-defined or on-demand reporting cycle such as hourly, weekly, or monthly. The reports can detail, in some embodiments, the exact location of each kit 300 and the trays 304 therein via respective parent trackers 308 and child trackers 310. Moreover, the instrument kit tracking system 300 and method of the present invention can record and transmit, within the same daily schedule, all date stamps, locations, and number of surgeries the kit 300 and the trays 304 associated with it has been involved in. A cloud-based system is preferably included to capture data on a daily basis and report out to the user or manager the particular status of each deployed kit 300, the trays 304 therein, namely, the medical instruments 306 being carried therein. The cloud-based system can additionally alert end users of any kit 300 of trays 304 that may have reached a predefined limitation on the number of surgeries performed so that the kit 300 can be returned, and instruments 306 replaced or calibrated before going back out for deployment. The ability to track trays 304 individually provides more fine tracking and monitoring of the entire tracking and monitoring of the present invention.

In the present embodiments the number of surgeries can recorded and tracked in many different ways. For example, the invention can record and track the surgeries by tracking the temperature of the device using a built-in temperature sensor. The cycling of temperature from a low, or nominal, temperature to a high temperature can be parsed, or understood, to represent a disinfecting wash cycle and a separate autoclave cycle, which is helpful in better understanding the compliance and usage information of the device and the instruments therein. For example, the average low temperature can be representative of transport to and usage during a surgical procedure. Other tracking methodologies could be used, such as pressure or an accelerometer, and any other methodologies that can carry out recording the number of surgeries. The instrument kit tracking system 300 and method of the present invention offers greater efficiency in inventory management, deployment, accountability of the assets, automatic annual inventory auditing, complete compliance to instrument usage and documentation, and ease of inventory recovery during product recalls. This is particularly true where individual trays 304 can be tracked using child trackers 310 that communicate with one or more parent trackers 308 nearby.

It will be appreciated by those skilled in the art that various changes and modifications can be made to the illustrated embodiments without departing from the spirit of the present disclosure. All such modifications and changes are intended to be covered by the appended claims.

What is claimed is:

1. A tracking system for a medical instrument kit, comprising:
an instrument kit housing;
a parent tracker affixed to the instrument kit housing; the parent tracker including a power source, a location sensor, and a temperature sensor;
at least one medical instrument tray residing in the instrument kit housing; the at least one medical instrument tray being configured and arranged to receive at least one medical instrument;
a child tracker affixed to each of the at least one medical instrument tray; the child tracker being configured and arranged to communicate data to the parent tracker;
wherein the temperature sensor in the parent tracker is configured and arranged to record a temperature reading and calculate a usage of the at least one medical instrument if the temperature reading is above a predetermined value.

2. The tracking system of claim 1, wherein the tracking system is configured and arranged to withstand external temperatures between 120-135° C.

3. The tracking system of claim 1, wherein the child tracker communicates data to the parent tracker if the child tracker is in wireless communication range of the parent tracker.

4. The tracking system of claim 1, wherein the internal temperature of the parent tracker is maintained such that an electronic device contained therein is operable when an external temperature is between 120-135° C.

5. The tracking system of claim 1, wherein the parent tracker and the child tracker are standalone units.

6. The tracking system of claim 1, wherein access to the parent tracker and child tracker are respectively selectively lockable.

7. The tracking system of claim 1, wherein the predetermined value is representative of an autoclave cycle.
